SEQUENCE LISTING (7 sequences)

SEQ ID NO: 1 - DNA, 1840 bp, Homo sapiens
gcggcgcgagccacatgcgagcgggcgcctggcggcggcggcggcggcaccagcgatcccagcagcggccacgacgcggacgcgcctgcggcccggggagcagcagcagccacagccacagcagccgccactgcagttagagcggcagcagcagcgacagccacagcagcagccgccgcggagagcggcgctcggcgggcgcgccctcctgaaggaagccgcccgccccccaccgccgccccctccggcgtgttcatgcccccggggccccagggagcgccatggcccgcgcacgccaggagggcagctccccggagcccgtagagggcctggcccgcgacggcccgcgccccttcccgctcggccgcctggtgccctcggcagtgtcctgcggcctctgcgagcccggcctggctgccgcccccgccgcccccaccctgctgcccgctgcctacctctgcgcccccaccgccccacccgccgtcaccgccgccctggggggttcccgctggcctgggggtccccgcagccggccccgaggcccgcgcccggacggtcctcagccctcgctctcgctggcggagcagcacctggagtcgcccgtgcccagcgccccgggggctctggcgggcggtcccacccaggcggccccgggagtccgcggggaggaggaacagtgggcccgggagatcggggcccagctgcggcggatggcggacgacctcaacgcacagtacgagcggcggagacaagaggagcagcagcggcaccgcccctcaccctggagggtcctgtacaatctcatcatgggactcctgcccttacccaggggccacagagcccccgagatggagcccaattaggtgcctgcacccgcccggtggacgtcagggactcggggggcaggcccctcccacctcctgacaccctggccagcgcgggggactttctctgcaccatgtagcatactggactcccagccctgcctgtcccgggggcgggccggggcagccactccagccccagcccagcctggggtgcactgacggagatgcggactcctgggtccctggccaagaagccaggagagggacggctgatggactcagcatcggaaggtggcggtgaccgagggggtggggactgagccgcccgcctctgccgcccaccaccatctcaggaaaggctgttgtgctggtgcccgttccagctgcaggggtgacactgggggggggggggctctcctctcggtgctccttcactctgggcctggcctcaggcccctggtgcttccccccctcctcctgggagggggcccgtgaagagcaaatgagccaaacgtgaccactagcctcctggagccagagagtggggctcgtttgccggttgctccagcccggcgcccagccatcttccctgagccagccggcgggtggtgggcatgcctgcctcaccttcatcagggggtggccaggaggggcccagactgtgaatcctgtgctctgcccgtgaccgccccccgccccatcaatcccattgcataggtttagagagagcgacgtgtgaccactggcattcatttggggggtgggagattttggctgaagccgccccagccttagtccccagggccaagcgctggggggaagacggggagtcagggagggggggaaatctcggaagagggaggagtctgggagtggggagggatggcccagcctgtaagatactgtatatgcgctgctgtagataccggaatgaattttctgtacatgtttggttaattttttttgtacatgatttttgtatgtttccttttcaataaaatcagattggaaca

SEQ ID NO: 2 - protein, 193 aa, Homo sapiens
MARARQEGSSPEPVEGLARDGPRPFPLGRLVPSAVSCGLCEPGLAAAPAAPTLLPAAYLCAPTAPPAVTAALGGSRWPGGPRSRPRGPRPDGPQPSLSLAEQHLESPVPSAPGALAGGPTQAAPGVRGEEEQWAREIGAQLRRMADDLNAQYERRRQEEQQRHRPSPWRVLYNLIMGLLPLPRGHRAPEMEPN

SEQ ID NO: 3 - protein, 131 aa, Homo sapiens
MKFGMGSAQACPCQVPRAASTTWVPCQICGPQPSLSLAEQHLESPVPSAPGALAGGPTQAAPGVRGEEEQWAREIGAQLRRMADDLNAQYERRRQEEQQRHRPSPWRVLYNLIMGLLPLPRGHRAPEMEPN

SEQ ID NO: 4 - protein, 101 aa, Homo sapiens
MKFGMGSAQACPCQVPRAASTTWVPCQICETRGAAAAPPLTLEGPVQSHHGTPALTQGPQSPRDGAQLGACTRPVDVRDSGGRPLPPPDTLASAGDFLCTM

SEQ ID NO: 5 - protein, 21 aa, Homo sapiens
AREIGAQLRRMADDLNAQYER

SEQ ID NO: 6 - protein, 8 aa, Artificial sequence
  Synthetic construct
  misc_feature (2)..(3): Xaa can be any naturally occurring amino acid
  MISC_FEATURE (4)..(4): X is L, I, or M
  MISC_FEATURE (5)..(5): X is L, C, G, A, or S
  MISC_FEATURE (7)..(7): X is R, E, D, K, or Q
  MISC_FEATURE (8)..(8): X is D, Y, N, H, or V
LXXXXDXX

SEQ ID NO: 7 - DNA, 926 bp, Artificial sequence
  Plasmid
gtcgtaataaccccgccccgttgacgcaaatgggcggtaggcgtgtacggtgggaggtctatatagcagagctcgtttagtgaaccgtcagaattaattcagatcttcaaaagaaacggaatggaaagctatgagacaggatgaaatttggcatggggtctgcccaggcatgtccatgccaggtgcccagggctgcttccacgacgtgggtcccctgccagatttgtggtcctcagccctcgctctcgctggcggagcagcacctggagtcgcccgtgcccagcgccccgggggctctggcgggcggtcccacccaggcggccccgggagtccgcggggaggaggaacagtgggcccgggagatcggggcccagctgcggcggatggcggacgacctcaacgcacagtacgagcggcggagacaagaggagcagcagcggcaccgcccctcaccctggagggtcctgtacaatctcatcatgggactcctgcccttacccaggggccacagagcccccgagatggagcccaattaggtgcctgcacccgcccggtggacgtcagggactcggggggcaggcccctcccacctcctgacaccctggccagcgcgggggactttctctgcaccatgtagcatactggactcccagccctgcctgtcccgggggcgggccggggcagccactccagccccagcccagcctggggtgcactgacggagatgcggactcctgggtccctggccaagaagccaggagagggattagcggccgcatcgatgatatcaagcttgcatgcctgcaggtcgactctagaggatccccgggtggcatccctgtgacccctccccagtgcctctcctggccttggaagttgccactccagtgcccaccagccttgtcctaataaaattaagttgcatc

(19) United States
(12) Patent Application Publication
(10) Pub. No.: US 2007/0065421 A1
(43) Pub. Date: Mar. 22, 2007

(54) Inducing expression of puma to reduce joint inflammation in the treatment of arthritis

(76) Inventors: Gary S. Firestein, Del Mar, CA (US); David L. Boyle, Santee, CA (US)

Correspondence Address: DLA PIPER US LLP, 4365 EXECUTIVE DRIVE, SUITE 1100, SAN DIEGO, CA, 92121-2133, US

(21) Appl. No.: 11/522,026

(22) Filed: Sep. 15, 2006

Related U.S. Application Data
(60) Provisional application No. 60/717,921, filed on Sep. 16, 2005.

(51) Int. Cl. A61K 48/00; A61K 38/54

(52) U.S. Cl. 424/93.21; 424/94.2; 514/44

(57) ABSTRACT

It has been discovered the BH3-only proapoptotic Bcl-2 proteins like PUMA (the p53 upregulated modulator of apoptosis) can be activated in fibroblast-like synoviocytes present in the joints of subjects with rheumatoid arthritis. This bypasses the p53 apoptotic pathway, which normally clears FLS fomr the joints, but is often defective in arthritis. Suitable therapeutic agents of this invention include gene vectors that cause increased expression of BH3-only proapoptotic proteins in target cells. These agents are formulated in pharmaceutical compositions for administration directly to joint. Reestablishing apoptosis in synoviocytes decreases their pro-inflammatory and destrcutive activity, improving the clinical condition.

### RELATED APPLICATIONS

This application claims the priority benefit of U.S. provisional application No. 60/717,921, filed Sep. 16, 2005.

For purposes of prosecution and interpretation in the U.S., the priority application is hereby incorporated herein by reference in its entirety

### TECHNICAL FIELD

This invention relates generally to programmed cell death (apoptosis), and more specifically to treatment of joint inflammation by induction of p53 upregulated modulator of apoptosis (PUMA), or other BH3-only proapoptotic Bcl-2 protein.

### BACKGROUND

Rheumatoid arthritis is characterized by joint inflammation and synovial tissue hyperplasia and invasion into cartilage and bone.

Normal synovium is comprised of a superficial cellular layer made up of large cells with prominent interdigitating cytoplasmic processes. These cells form a lining one to three cells deep that rests on compact connective tissue bearing a vascular plexus and occasional cells. This superficial layer, which faces the joint cavity, is referred to as the synovial intimal or intimal lining. The lining is comprised of two types of synoviocytes that can be distinguished on

morphologic, histochemical, and immunohistologic characteristics. The type A synoviocytes, which account for approximately one-third of the cell lining the normal synovium and as many as 80% of lining cells in rheumatoid arthritis, have the characteristics of monocytes or terminally differentiated resident tissue macrophages.

The remaining cells (type B synoviocytes) have morphologic features of fibroblasts with a regular membrane, limited numbers of filopodia, and large amounts of rough endoplastic reticulum, consistent with active metabolic processes. With inflammation, the synovial membrane becomes markedly expanded, edematous, and infiltrated with a variety of cells. As a part of this process, the lining cells become redundant, increase in number, and participate in the formation of villous projections. In addition, synovial lining cells increase many hundred-fold, assume phenotypic features of malignant cells, and produce proteases and cytokines like IL-6 that exacerbate the inflammatory process. These cells have a direct pathological effect on cartilage, and recruit other effector cells such as osteoclasts that then degrade bone.

Several therapeutic approaches to rheumatoid arthritis have focused on restoring apoptosis in the synovium, especially the intimal lining. The accumulation of cells in the lining can be due to ingress of cells form the blood, local proliferation, or insufficient deletion through apoptosis.

Cellular accumulation associated with rheumatoid arthritis and osteoarthritis correlates with where apoptosis occurs in situ. Genomic DNA extracted from rheumatoid arthritis synovium demonstrated DNA ladders characteristic of apoptosis. An in situ end labeling assay using digoxigenin-labeled nucleotides and alkaline phosphatase-labeled antibody identified DNA strand breaks in frozen synovial tissue sections from patients with rheumatoid arthritis. Although the number of DNA strand breaks observed was increased in the synovial tissue from rheumatoid arthritis patients relative to normal and osteoarthritis controls, unexpectedly, a smaller than normal percentage of cells with DNA strand breaks were apoptotic. Apoptotic cells were primarily in the synovial lining and were predominantly macrophages, although fibroblast-like cells also had evidence of DNA fragmentation.

Accordingly, it has been proposed that synovial lining expands in the joint from recruitment of macrophage-like and fibroblast-like cells and deficiencies in the apoptosis pathway. Apoptosis of the synovial cells in rheumatoid arthritis appears to be an active process that involves both macrophage-like and fibroblast-like cells and is likely enhanced by the local cytokine milieu, regional production of reactive oxygen species, and intermittent ischemia and reperfusion in the joint. Despite the evidence for abundant DNA fragmentation consistent with apoptosis, it appears that this process is not rapid enough to maintain the normal synovial lining thickness since synovial lining expansion does, in fact, exist in rheumatoid arthritis. To account for this insufficiency of apoptosis, it is believed that the apoptosis pathway might be defective or aberrant, and that cells with DNA strand breaks might either recover or persist for prolonged periods of time. (Yamanishi, Firestein et al., Rheum Dis Clin North Am. 2001 May;27(2): 355-71; Arthritis Res Ther. 2005;7(1):R12-8)

Several genes have been implicated in impairment of the apoptosis pathway p53, which is thought to play an especially important role in cell survival, is a transcription factor that regulates cell cycle progression, DNA repair, proliferation, and programmed cell death (apoptosis). In fact, as much as 10% of the cDNA pool from inflamed synovial tissue may have mutations in the p53 gene.

U.S. Pat. Nos. 6,004,942 and 6,747,013 (Firestein et al.) describe methods for treating arthritis by administering agents that induce or regulate apoptosis. Patents are treated with a nucleic acid molecule encoding a protein that enhances apoptosis in the apoptosis defective cells. Model agents include p53, ICE, bax, p21 waf ras, and Fas ligand.

Subsequently, Yao et al. (Mol Ther. 2001 Jun;3(6):901-10) reported that gene transfer of p53 to arthritic joints stimulates synovial apoptosis and inhibits inflammation. Adriaansen, Firestein et al. (Ann Rheum Dis. 2005 Dec;64(12):1677-84) treated arthritis by intra-articular dominant negative IKKβ gene therapy using AAV. Yal et al. (Hum Gene Ther. 2006 Epub) found that adenoviral mediated delivery of Fas ligand to arthritic joints causes extensive apoptosis in the synovial lining. Zhang et al. (Arthritis Res Ther. 2005;7(6):R1235-43) reported elimination of rheumatoid synovium in situ using a Fas ligand ‘gene scalpel’. Takahashi et al. (Clin Exp Rheumatol. 2005 Jul-Aug;23(4):455-61) tried AAV-mediated anti-angiogenic gene therapy for collagen-induced arthritis in mice. Nabbe et al. (Arthritis Res Ther. 2005;7(2):R392-401) reported that local IL-13 gene transfer prior to immune-complex arthritis inhibits chondrocyte death and matrix-metalloproteinase-mediated cartilage matrix degradation.

These and other studies demonstrate that the synovial space is amenable to gene therapy, leading to effective expression of the encoded protein. However, not all of the gene products under development have proved effective. In some studies, Fas ligand has turned out to be pro-inflammatory, while a dominant negative IKKβ product was found to have a deleterious effect on NF-κB regulated pathways. Meanwhile, some patients with osteoarthritis are currently being treated with 3-5 injections of hyaluronic acid per week, amounting to over $250 million in sales in the U.S. in the past year—even though the benefits are equivocal.

Accordingly, there is a pressing need to develop new agents for the treatment of rheumatoid arthritis, and other arthropathies.

### SUMMARY OF THE INVENTION

It has been discovered that BH3-only proapoptotic Bcl-2 proteins like PUMA (the p53 upregulated modulator of apoptosis) can be activated in fibroblast-like synoviocytes present in the joints of subjects with rheumatoid arthritis. This bypasses the p53 apoptotic pathway, which normally clears FLS from the joints, but is often defective in arthritis. Reestablishing apoptosis in synoviocytes decreases their pro-inflammatory and destructive activity, helping to resolve the clinical condition.

One aspect of this invention is a pharmaceutical composition comprising a BH3-only proapoptotic Bcl-2 protein, or a nucleic acid vector encoding said protein. The composition may be formulated in an excipient for administration into an inflamed joint, and intended for treating an inflammatory condition, such as rheumatoid arthritis or osteoarthritis. An exemplary BH3 only proapoptotic protein is human PUMA, and proapoptotic fragments and variants thereof, as described below. Exemplary expression vectors are based on adenovirus, optionally optimized to improve tropism or expression levels in fibroblast-like synoviocytes or other cells in the joint.

Further aspects of the invention include a method for preparing such pharmaceutical compositions, and the use of such compositions for killing cells such as fibroblast-like synoviocytes (FLS) in vitro. In the development stage, the invention includes a method for determining whether an agent can induce expression of a BH3-only proapoptotic Bcl-2 protein in FLS by combining them in vitro, and determining whether apoptosis is induced in said FLS as a consequence thereof, according to a suitable assay. Suitable candidates for screening include BH3 proteins and variants, nucleic vectors encoding them, small molecule drugs, and other agents that induce BH3 protein expression or activation. Once identified as effective in such assays, the agents can be used to prepare further pharmaceutical compositions of the invention. Such assays can also be used for quality control to assess the functionality and potential clinical effectiveness during ongoing production of such compositions.

Other aspects of the invention are treatment modalities and methods. Included are methods causing apoptosis in synoviocytes and other inflammatory cells in vivo, by administering an agent that causes expression or activation of BH3-only pro-apoptotic Bcl-2 proteins in the target cells. The agent is typically administered at or around the site of an inflamed joint in a human patient or other mammal, as part of a treatment for rheumatoid arthritis, osteoarthritis, or other condition that causes or is accompanied by joint inflammation. Again, a non-limiting example suitable for use in such therapies is the beta-isoform of human PUMA, encoded in an adenovirus vector.

Other aspects of the invention will be evident from the description that follows.

### BRIEF DESCRIPTION OF THE DRAWINGS

FIG. 1 compares the three isoforms of human PUMA (SEQ. ID NOs:2-4), and shows the location of the BH3 domain (SEQ. ID NO:5) with characteristics of the BH3 motif (SEQ. ID NO:6).

FIG. 2 shows a plasmid containing the PUMA-β gene (SEQ. ID NO:7) used to assemble an Adenoviral Type 5 vector in E. coli.

FIG. 3 shows endogenous PUMA protein and gene expression in synovial tissue (ST) from rheumatoid arthritis (RA) or osteoarthritis (OA), and in fibroblast-like synoviocytes (FLS). FIG. 3(A): Western blot analysis. FIG. 3(B): Immunohistochemistry of synovial tissue. FIG. 3(C): Quantitative PCR analysis of PUMA expression at the mRNA level in different tissues.

FIG. 4 shows induction of p21 but not PUMA by an adenovirus expression vector for the pro-apoptotic gene p53. FIG. 4(A): Western blot for protein expression. FIG. 4(B): Quantitative comparison showing that Ad-p53 increased p21 protein expression but not PUMA. FIG. 4(C): Failure of arthritis FLS to respond to reactive oxygen or the nitric oxide donor SNAP.

FIG. 5 shows that apoptosis was induced in FLS by PUMA overexpression. FIG. 5(A): Trypan Blue exclusion. FIG. 5(B): Comparison of apoptosis induced by pCEP4, HA-PUMA, or HA-PUMAdBH3 in RA (closed bar) and OA (open bar) FLS lines. FIG. 5(C): Significant DNA fragmentation (determined by ELISA) was seen in HA-PUMA-transfected cells compared with pCEP4-transfected cells. FIG. 5(D): Effect of gene transduction on caspase-3 activation (determined by immunostaining). PUMA-transfected FLS showed significantly higher activation of caspase-3.

FIG. 6 shows the effect of siRNA p53 knockdown on cultured FLS. FIG. 6(A): Western blot analysis with siRNA or non-silence scrambled siRNA (sc). FIG. 6(B): Immunohistochemistry staining of p53 protein expression in RA FLS. Immunohistochemistry also demonstrated a marked decrease in the percentage of p53 positive cells after siRNA transduction.

FIG. 7 shows the effect of p53 siRNA on FLS function. FIG. 7(A): cell growth. FIG. 7(B): p21 protein expression. These data indicate that p53 knockdown leads to functional alterations consistent with p53 deficiency.

FIG. 8 is taken from an experiment conducted to determine if p53 is required for PUMA-induced apoptosis. Cultured p53-deficient human FLS were transfected with siRNA and then with 10 μg of PUMA, PUMAdBH3, or pCEP4. FIG. 8(A):. DNA fragmentation, as determined by histone release. FIG. 8(B): Cell viability at 24 hr after cDNA transfection of siRNA-treated FLS. Mock (stippled square), PUMA (black square), PUMAdBH3 (gray square), and pCEP4 (open square). FIG. 8(C): Cell viability. PUMA (black square) and pCEP4 (open square), only. Histone release or cell viability were similar in scrambled siRNA- and p53 siRNA-transfected cells.

FIG. 9 illustrates the effect of PUMA on p53+/+and p53−/−murine FLS. FIG. 9(A): Cell viability. FIG. 9(B): PUMA-induced apoptosis (histone release) in both wild type and p53 knockout FLS. Overall cell viability was decreased in the transfected cells, but the effect of PUMA compared with PUMAdBH3 or empty vector

FIG. 10 illustrates the effect of PUMA on human FLS containing mutant p53. Human FLS were transfected with dominant negative p53 cDNA R213* or control, followed by PUMA cDNA. PUMA still effectively induced apoptosis in FLS.

### DETAILED DESCRIPTION

The underlining basis of the invention can be understood from the idea that fibroblast-like synoviocytes (FLS) present in arthritic joints fail to clear in the proper fashion because of a defect in apoptosis.

The p53 gene product (a Bcl-2 protein) is a key mediator of the apoptotic pathway. One effect of p53 is to stop proliferation of the cell, by way of p21 protein. The other effect is to initiate the cascade of events that leads to DNA fragmentation, mediated more directly by effector proteins such as Bax and Bak. The pathway is under tight regulatory control by a family of proteins with proapoptotic and inhibitory activity. For general reviews on the various players in the apoptotic pathway, the reader can refer to reviews by Labi et al., Cell Death Differ. 13:1325-38, 2006; Willis et al., Curr Opin Cell Biol. 17:617-25, 2005; Letai et al., Cancer cell 2:183-02, 2002; Juin et al., Cell Cycle 4:637-42, 2005; R.J. Lutz, Biochem Soc Trans. 28:51-56, 2000; and Petros et al., Biochim Biophys Acta. 1644:83-94, 2004. Modification of FLS in inflamed joints lead to mutations in not only p53, but also other apoptosis genes involved in the apoptosis pathway. Because the components interact in complex ways, it can be difficult to overcome all the functionai deficiencies, without inadvertently triggering other events with negative consequences.

This disclosure shows for the first time that activating BH3-only proapoptotic Bcl-2 proteins like PUMA can often sidestep all the defects, thereby restoring apoptosis to FLS, and helping to resolve the clinical condition.

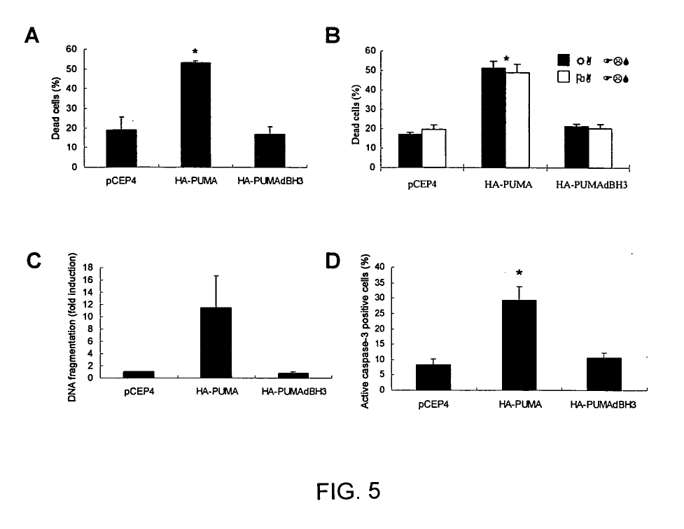

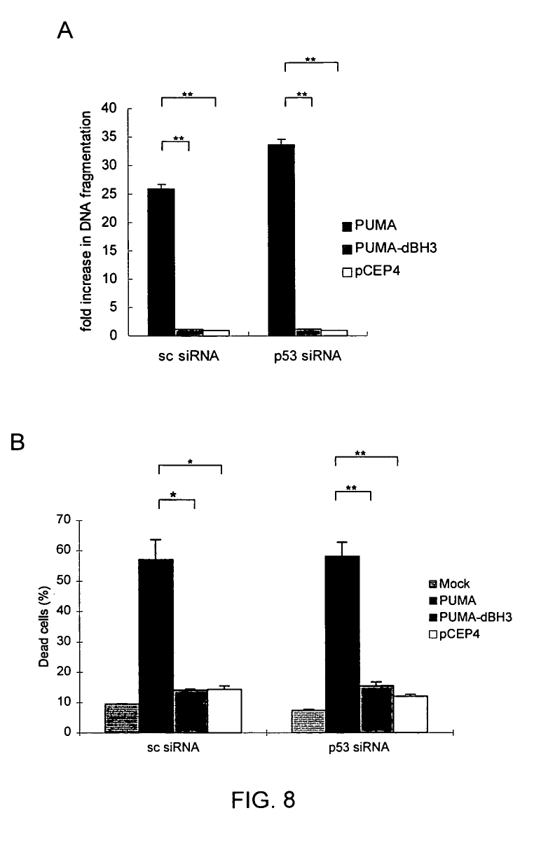

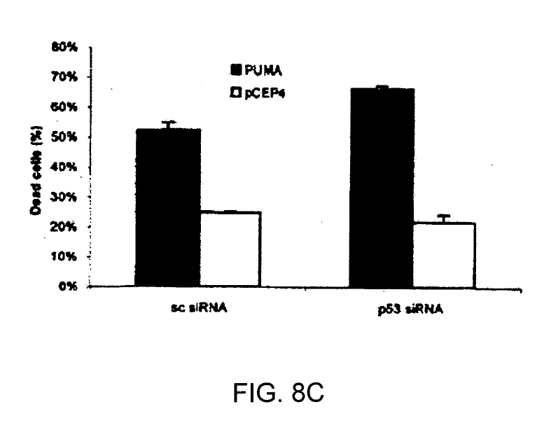

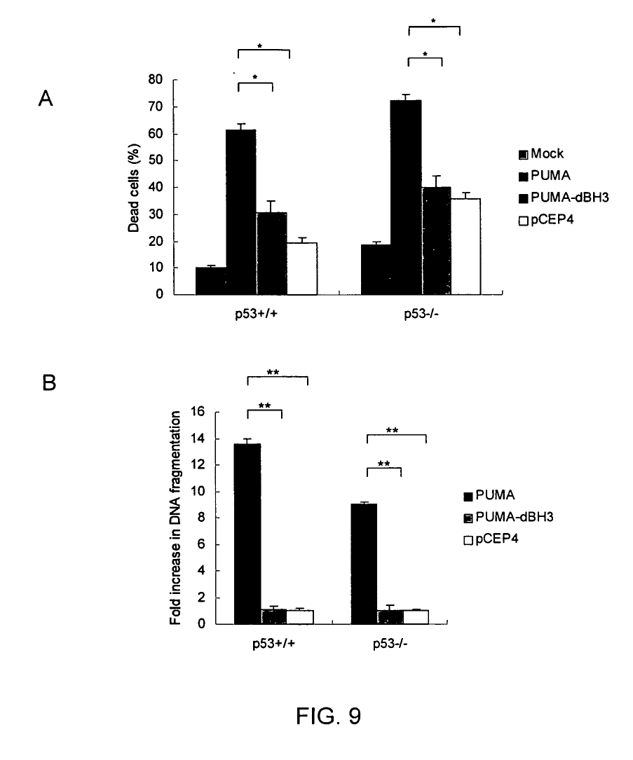

FIG. 5 provides evidence that apoptosis can be induced in FLS by PUMA overexpression, even though they were taken from inflamed synovium. Several hallmarks of apoptosis are present: decreased cell viability, DNA fragmentation, and activation of the effector caspase-3. FIG. 8 shows that active p53 protein need not be present for PUMA-induced apoptosis. Blocking p53 transduction with siRNA does not block the effectiveness of PUMA. Similarly, the data in FIG. 9 show that PUMA is effective in promoting apoptosis even in cells that are homozygous for inactivation of the p53 gene.

This discovery is contrary to the prevailing wisdom in the published literature.

Green et al. (Science. 2005 Sep. 9;309(5741):1732-5) has provided evidence that there is a nexus between Bcl-xL, cytoplasmic p53, and PUMA that coordinates the distinct functions of p53. They found that after genotoxic stress, Bcl-xL sequestered cytoplasmic p53. Nuclear p53 caused expression of PUMA, which then displaced p53 from Bcl-xL, allowing p53 to induce mitochondrial permeabilization. Mutant Bcl-xL that bound p53, but not PUMA, rendered cells resistant to p53-induced apoptosis irrespective of PUMA expression. This implies that PUMA is insufficient to initiate apoptosis in the absence of functioning p53.

The surprising effectiveness of BH3-only proapoptotic proteins in restoring apoptosis to fibroblast-like synoviocytes foreshadows a new era in the treatment of rheumatoid arthritis. A currently available modality for severe joint pain is corticosteroid injections. Steroids reduce inflammation, but do not clear out FLS from the joints, meaning that inflammation may reoccur in a matter of weeks. On the other hand, synovectomy has longer lasting effects (a few years), but comes with the expense and inconvenience of a surgical procedure. The proapoptotic therapy of this invention is effectively a microsurgery technique using an off-the-shelf product —potentially combining the non-invasiveness of steroid injections, with the long-term relief of surgery.

The description of the underlying theory of how the pharmaceutical compositions are believed to work is provided here for the edification and enjoyment of the reader, without intending to limit how the invention is practiced. Many of the compositions described here are designed to increase expression of PUMA or another BH3-only protein in fibroblast-like synoviocytes, thereby promoting apoptosis and clearance of the FLS from the inflamed synovium. However, except where explicitly required, the compositions may or may not have all the properties that are forecast. By way of illustration, a nucleic acid vector of this invention may target other cells in addition or instead of FLS, and the resulting gene expression and apoptosis may or may not be measurable. The agent may promote clearance of FLS or reduce inflammation, regardless of whether the FLS were genetically defective or impaired before treatment. There is no need for the clinician or patient to monitor these things, as long as the composition is at least partially effective for its intended purpose, which is typically to ameliorate symptoms or signs of an unfavorable condition.

BH3-only proapoptotic Bcl-2 proteins and assays

Some embodiments of this invention are pharmaceutical compositions comprising a purified BH3-only proapoptotic protein, or a nucleic acid vector encoding such protein.

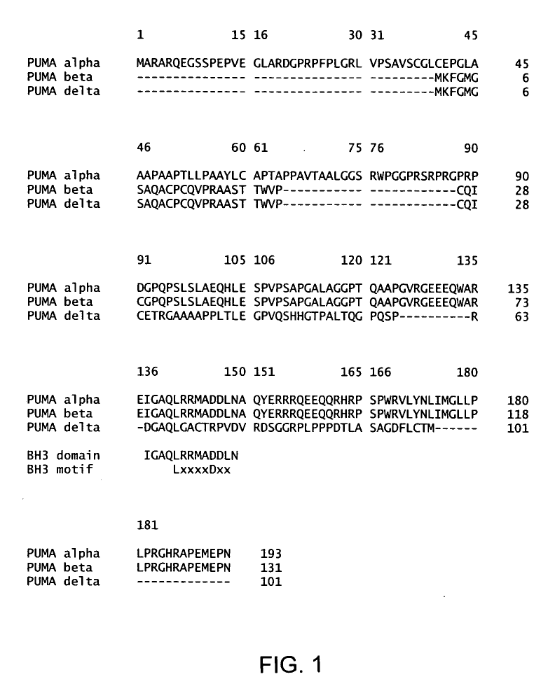

Exemplary is PUMA (the p53 upregulated modulator of apoptosis; also known as the Bcl-2 binding component 3; bbc3). As shown in FIG. 1, the alpha (GenBank AF354654.1 and NM_{—}014417; SEQ. ID NO:2) and beta (AF354655.1; SEQ. ID NO:3) splice variant isoforms of human PUMA both have the BH3 domain sequence IGAQLRRMADDLN (i.e., residues 4-16 as set forth in SEQ. ID NO:5). The human PUMA sequence has been determined by Yu, Vogelstein et al. (Mol Cell. 2001 Mar.;7(3):673-82) and by Nakano et al. (Mol Cell. 2001 Mar;7(3):683-94). The effectiveness of human PUMA-β in causing apoptosis of fibroblast-like synoviocytes from inflamed human joint tissue is illustrated in Examples 2 and 3.

It is believed that many (if not all) of the other native BH3-only proapoptotic Bcl-2 proteins are suitable as alternatives to PUMA. Effective proapoptotic proteins can be identified without undue experimentation using a suitable assay for apoptosis.

Apoptosis assays can be run using any suitable culture of mammalian cells (e.g., skin fibroblasts or established cell lines). Especially suitable are cultures of human fibroblast-like synoviocytes (FLS), which can be established from tissue taken from patients with rheumatoid arthritis or osteoarthritis at the time of joint replacement (Example 1); or FLS cell lines transduced with telomerase reverse transcriptase (Sun et al, Biochem Biophys Res Commun. 2004 Oct. 29;323(4):1287-92). The cultured FLS are contacted with an agent (such as a nucleic acid vector) that causes expression of BH3-only proapoptotic protein, and apoptosis is determined.

For the purposes of high throughput screening, extent of apoptosis can be determined by Trypan Blue exclusion. Briefly, 10 μg of plasmid is transfected into human FLS derived from patients undergoing joint replacement for osteoarthritis, using Amaxa™ electroporation. The next day (22-24 h later), adherent cells are harvested by trypsinization and pooled with floating cells, and an aliquot stained with Trypan Blue for live/dead cell differentiation by light microscopy. The protein will be “proapoptotic” if there is substantially higher proportion of stained cells (preferably at least 20%) compared with control. For more detailed analysis, the protein can be tested at serial dilutions, and the effect can be determined by other events that are linked to apoptosis—such as ELISA for histone proteins (Example 1) DNA fragmentation (Example 3) or the activation of apoptosis related genes like caspase-3.

The terms “BH3-only proapoptotic Bcl-2 protein” or more simply “BH3 protein” as used in this disclosure mean a protein that contains the third amphipathic homology (BH) domain characteristic of the Bcl-2 family (Huang and Strasser, Cell (2000) 103:839-842), and which has the function of supporting or promoting apoptosis when expressed in mammalian cells. In some embodiments, artificial constructs or fragments comprising a BH3 domain may be included (for example, US 2005/0064593 Al). BH3 proteins have a BH3 domain with the amino acid motif LX₁X₂X₃X₄DX₅X₆ (SEQ. ID NO:6), with the following criteria:
X₁, X₂ are any amino acid
X₃ is a nonpolar amino acid selected from L, I, and M
X₄ is an amino acid with a short side chain, such as L, C, G, A, or S
X₅ is a charged or polar amino acid such as R, E, D, K, or Q
X₆ is usually a polar amino acid, exemplified by but not limited to D, Y, N, H or V.
   BH3-only Proapoptotic Bcl-2 Protein Variants

Included in the family of BH3 proteins that can be used in this invention are artificial fragments, variants, and fusion proteins that are structurally modeled on PUMA and other native mammalian BH3 proteins, which retain the functional ability of the native counterparts to promote or support apoptosis when expressed in a cell.

To obtain BH3 proteins that are fragments of native BH3 proteins, the native protein is trimmed at the N—or C-terminus (either by proteolysis or by recombinant expression), and then tested for pro-apoptotic activity. Trimming may continue until activity is lost, at which point the minimum functional unit would be identified. Fragments containing any portion of the native protein down to the minimum size are expected to be functional, as would fusion constructs containing at least the functional core of the protein, with additional amino acids at either end.

Protein variants can be generated by recombinant expression, mutating the nucleic acid sequence encoding a native BH3 protein or fragment thereof so as to induce one or more amino acid changes in the encoded protein. One approach is to perform site-specific mutagenesis guided by known homology data and its imputed role in protein function—for example, avoiding mutations in the BH3 domain. Adopting this strategy, the user would obtain a homolog identifiable by a degree of sequence identity (or an ability of the gene sequence to hybridize with the prototype nucleic acid sequence), which could then be tested for pro-apoptotic activity.

However, unless particular changes are desired, there is no need to target the mutations to particular positions in the sequence. An effective way to generate a large collection of functional variants is to use a random mutation strategy. The standard texts Protocols in Molecular Biology (Ausubel et al. eds.) and Molecular Cloning: A Laboratory Manual (Sambrook et al. eds.) describe techniques employing chemical mutagenesis, cassette mutagenesis, degenerate oligonucleotides, mutually priming oligonucleotides, linker-scanning mutagenesis, alanine-scanning mutagenesis, and error-prone PCR. Other efficient methods include the E. coli mutator strains of Stratagene (Greener et al., Methods Mol. Biol. 57:375, 1996) and the DNA shuffling technique of Maxygen (Patten et al., Curr. Opin. Biotechnol. 8:724, 1997; Harayama, Trends Biotechnol. 16:76, 1998; U.S. Pat. Nos. 5,605,793 and 6,132,970). To increase the extent of variation, the user may subject the prototype sequence to successive cycles of mutation and functional testing—or choose a mutation strategy that generate more abrupt changes, such as the DNA shuffling technique.

There are several commercially available services and kits available to the skilled reader to use in obtaining variants of the claimed proteins: for example, the GeneTailor™ Site-Directed Mutagenesis System sold by InVitrogen™ Life Technologies; the BD Diversify™ PCR Random Mutagenesis Kit™, sold by BD Biosciences/Clontech; the Template Generation System™, sold by MJ Research Inc., the XL1-Red™ mutator strain of E. coli, sold by Stratagene; and the GeneMorph® Random Mutagenesis Kit, also sold by Stratagene. By employing any of these systems in conjunction with an assay for apoptosis, variants can be generated and tested in a high throughput manner. The user then has the option of subjecting the variant clones to further rounds of mutagenesis, until the desired degree of variation from the native sequence has been achieved.

Using techniques such as those described here variants, and fusion proteins can be obtained that contain a number of alterations, additions, or deletions. Variants can be obtained that are at least 80%, 90%, 95%, 98%, or 100% identical or homologous to a prototype naturally occurring BH3 protein. The length of the identical or homologous sequence compared with the native protein can be about 30, 50, 100, or 200 residues, up to the length of the full-length protein. Unless explicitly defined otherwise, the term “PUMA” used in this disclosure includes native proapoptotic isoforms of human PUMA, PUMA from other mammalian species, and artificial fragments and variants of such native forms. Embodiments of this invention exemplified by PUMA can generally be practiced mutatis mutandis using other BH3 proteins.

Pharmaceutical compositions

Compositions for pharmaceutical use according to this invention are designed to promote apoptosis in pro-inflammatory cells that have accumulated in or around joints or other inflammatory tissue, by activating a BH3 proapoptotic gene in the target cell population

One approach is to use a nucleic acid vector that induces expression of the protein in the target cell (illustrated in Examples 2 and 3). The term “nucleic acid vector” as used in this disclosure means a nucleic acid (DNA or RNA) with the encoding sequence, and having other components (e.g., other nucleic acid sequences, associated proteins and/or capsids) that allow the encoding sequence to be expressed in the target cell.

Suitable vectors include DNA plasmids (Example 1), optionally associated with transfection assisting agents such as Lipofectamine 2000™ or FuGENE™. For use in human therapy, adenoviral vectors are especially suitable because there is no integration, transfection is temporary, they can be replication deficient, and the target cells need not be proliferating. Adeno-associated virus (AAV), herpes virus, vacciniavirus, retroviruses, or other viral vectors may be used if they have appropriate tropism for cells in the synovium (e.g., fibroblast-like synoviocytes and macrophages). Reconstituted Sendai virus envelopes are also suitable as intra-articular drug vectors: (Earl et al., J Pharm Pharmacol. 1988 Mar;40(3):166-70).

One approach for introduction of nucleic acid into a cell is by use of a viral vector containing nucleic acid, encoding a BH3 only proapoptotic Bcl-2 protein. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors can be used as a recombinant gene delivery system for the transfer of exogenous nucleic acid molecules encoding BH3 only proapoptotic Bcl-2 protein in vivo or ex vivo. These vectors provide efficient delivery of nucleic acids into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, A. D. (1990) Blood 76:271). A replication defective retrovirus can be packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10 9.14 and other standard laboratory manuals.

Another viral gene delivery system useful in the present invention uses adenovirus-derived vectors. The genome of an adenovirus can be manipulated such that it encodes and expresses a gene product of interest but is inactivated in terms of its ability to replicate in a normal lytic viral life cycle. See, for example, Berkner et al. (1988) BioTechniques 6:616; Rosenfeld et al. (1991) Science 252:431 434; and Rosenfeld et al. (1992) Cell 68:143 155. Suitable adenoviral vectors derived from the adenovirus strain Ad type 5 dl 324 or other strains of adenovirus (e.g., Ad2, Ad3, Ad7 etc.) are known to those skilled in the art. Recombinant adenoviruses can be advantageous in certain circumstances in that they are not capable of infecting nondividing cells and can be used to infect a wide variety of cell types, including epithelial cells (Rosenfeld et al. (1992) cited supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267).

Another viral vector system useful for delivery of the subject nucleotide sequence encoding BH3 only proapoptotic Bcl-2 protein is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97 129). It is also one of the few viruses that may integrate its DNA into non-dividing cells, and exhibits a high frequency of stable integration (see for example Flotte et al. (1992) Am. J. Respir. Cell. Mol. Biol. 7:34 9356; Samulski et al. (1989) J Virol. 63:3822 3828; and McLaughlin et al. (1989) J. Virol. 62:1963 1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251 3260 can be used to introduce DNA into cells. A variety of nucleic acids have been introduced into different cell types using AAV vectors (see for example Hermonat et al. (1984) Proc. Natl. Acad. Sci. USA 81:6466 6470; Tratschin et al. (1985) Mol. Cell. Biol. 4:2072 2081; Wondisford et al. (1988) Mol. Endocrinol. 2:32 39; Tratschin et al. (1984) J. Virol. 51:611 619; and Flotte et al. (1993) J. Biol. Chem. 268:3781 3790).

Any of a variety of promoters can be used to drive gene expression, including but not limited to constitutive promoters (e.g., cytomegalovirus, adenovirus, or SV40), promoters specific for the target tissue (e.g., gliostatin/platelet-derived endothelial cell growth factor for fibroblast-like synoviocytes; CD14 or CD68 for macrophages), and promoters potentially upregulated in arthritis (e.g., cytokines like IL-1, TNFa, and IL-6, or metalloproteinases). In some embodiments of the invention, a nucleic acid sequence encoding a BH3 protein is operatively linked to a suitable promoter and packaged in a vector for delivery into the target tissue. As an alternative, a nucleic acid vector for causing expressing a BH3 protein in a tissue will not encode the protein, but will induce protein expression by other means upon transduction. For example, the endogenous promoter that normally controls expression of the BH3 gene in the target cells can be functionally replaced with a heterologous promoter that causes the level of expression to increase. See U.S. Pat. Nos. 6,270,989 and 6,565,844 (Transkaryotic Therapies).

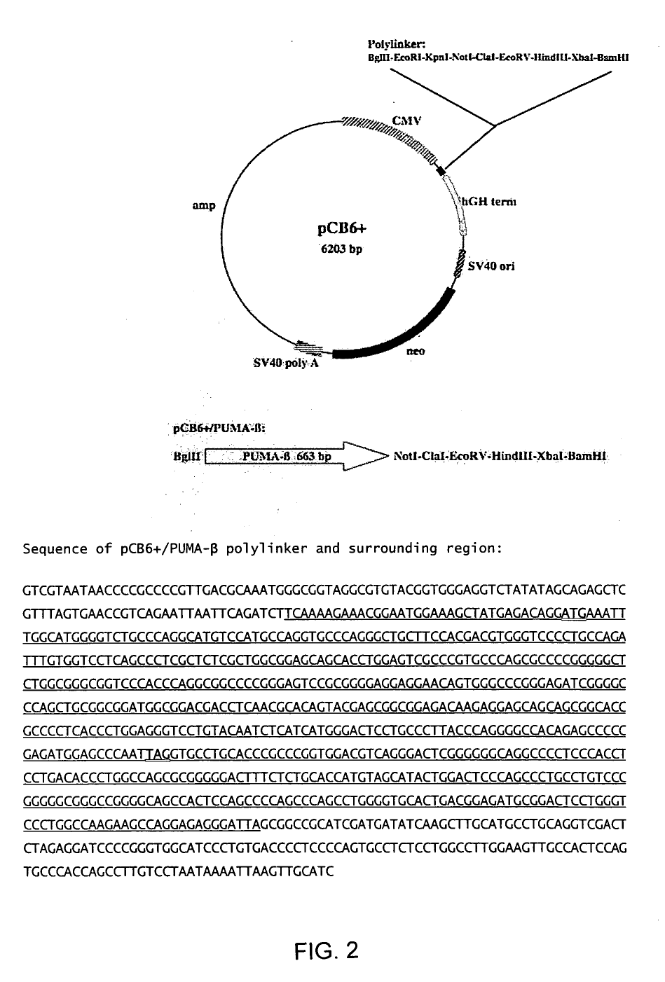

An exemplary adenovirus vector for initial testing can be made by such commercial services as Qbiogene (MP Biomedicals, Irvine CA) by supplying the human PUMA-βgene. The vector is assembled using the Adenovator™ CMV virus system, involving homologous recombination in E. Coli between a transfer vector and a plasmid containing the E1/E3 deleted genome of Ad5 (Adenovirus type 5; ATCC VR-5). The gene encoding the BH3 protein is cloned (FIG. 2), inserted into a transfer vector, and then co-transformed with the Ad5-backbone containing plasmid into BJ5183 E. coli. Clones can be assayed for high titers using the FLS apoptosis assay, as described earlier. Negative control can be an empty vector, or a vector containing a homolog of the BH3 protein with the BH3 domain deleted (Example 2).

Testing with cultured cells and in animal models for arthritis allows the user to reselect or optimize the vector to increase tropism for the target cell type, to increase transduction efficiency, and to minimize immunogenicity. Adenovirus constructs optimized for arthritis treatment are described by Yu et al. (Chin Med J (Engl). 2006 Aug. 20;119(16):1365-73), Toh, van den Berg, et al. (J Immunol. 2005 Dec. 1;175(11):7687-98) and others. AAV constructs optimized for arthritis treatment are described by Hirade et al. (Hum Gene Ther. 2005 Dec.;16(12):1413-21), Jorgensen et al. (Ann Rheum Dis. 2005 Dec.;64(12):1677-84), and Adriaansen et al. (Ann Rheum Dis. 2005 Dec.;64(12): 1677-84). A vector is referred to as having “improved tropism” for FLS or macrophages if it is at least 1.5-fold (and preferably at least 3-fold) more efficient or more selective for FLS than wild-type vector.

Another approach for mobilizing BH3 protein in the treatment of arthritis is to administer recombinant protein directly into the joint. Recombinant protein can be produced by expression cloning in prokaryotes such as E. coli (ATCC Accession No. 31446 or 27325), eukaryotic microorganisms such as Pichia pastoris yeast:, or higher eukaryotes, such as insect or mammalian cells (U.S. Pat. No. 5,552,524), and then purified using standard protein separation techniques.

To enhance entry of the protein into cells, it may be helpful to adapt the protein with a component that enhances membrane penetration, such as a protein membrane transducing domain (e.g., the HIV Tat protein: Blackwell et al., Arthritis Rheum. 50:2381-2386, 2004) or a lipid component (covalently attached, or as part of a liposome composition).

A further approach for activating BH3 protein activity in the target cell is with an oligonucleotide or small molecule drug that activates the endogenous promoter or otherwise increases transcription from the BH3 protein gene in the cell. Simoes-Wust et al. (J Neurooncol. 2005 Mar.;72(1):29-34) have reported that DeltaNp73 antisense activates PUMA and induces apoptosis in neuroblastoma cells.

Other effective agents can be identified using a method for determining whether an agent that promotes expression of a BH3-only proapoptotic Bcl-2 protein is suitable for preparation of a medicament for treating an inflamed joint. For example, cultures of fibroblast-like synoviocytes can be treated with a test agent from a library of compounds and tested for apoptosis by Trypan Blue exclusion, as before. Alternatively, the cells can be transfected with a construct comprising the natural promoter for the target BH3 protein driving a heterologous reporter gene such as green fluorescent protein or luciferase. Pro-apoptotic activity caused by induction of BH3 protein will then correlate with increased activity of the reporter gene.

Once the effective agent has been identified and optimized, a pharmaceutical composition is prepared by formulating an effective dose of the agent for administration into an area of inflammation in a subject in need thereof. Medicaments intended for human administration will be prepared in adequately sterile conditions, in which the active ingredient(s) are combined with an isotonic solution or other pharmaceutical carrier appropriate for the recommended therapeutic use. This is readily accomplished by filtration through sterile filtration membranes, prior to or following lyophilization and reconstitution. Therapeutic compositions herein generally are placed into a container having a sterile access port, for example, a vial having a stopper pierceable by a hypodermic injection needle.

Depending on the nature of the active ingredient and its intended purpose, effectiveness of the composition can be enhanced by using a formulation especially tailored for administration to the target site. For administration intra-articularly, nucleic acid vectors of this invention can be selected for improved tropism for synoviocytes, as already described. The formulation can be adapted to keep the vector or active ingredient in the joint (by increasing particle size) while limiting penetration into the cartilage (e.g., by making the particle more negative).

The effective size of the agent can be increased for intra-articular administration by incorporating into a liposome, noisome, or lipogelosome, (Turker et al., Int J Pharm. 2005;296(1-2) :34-43). Alternatively, the agent can be incorporated into a biodegradable microsphere made of natural compounds such as albumin or chitosan (Bozdag et al., J Microencasul. 2001;18(4):443-456; Thakkar et al., J Drug Target. 2004;12(9-10):549-557; Lamerio et al., Biotechnol. 2006 Jun. 3 Epub); or synthetic polymers such as poly(lactide-co-glycolide) (PLGA), poly(L-lactic acid) (PLA) and poly(caprolactone) (PCL) (Liggins et al., Inflamm Res. 2004;53 (8):363-372; Puebla et al., J Microencapsul. 2005;22 (7):739-808) of about 30 to 150 μm in diameter. Where the agent is a nucleic acid vector, the effective particle size can be increased by non-covalent or reversible aggregation, or by attachment onto a degradable biopolymer such as fibrin (Breen et al., J Biomed Mater Res A. 2006;78A(4):702-708).

If desired, the composition may include one or more other active ingredients designed to improve the clinical condition of the subject. Such additional ingredients could include corticosteroids and/or non-steroidal anti-inflammatories (such as naproxen) for the treatment of rheumatoid arthritis; or chondroprotective drugs (such as pentosan polysulphate), viscosupplements (such as hyaluronan), and/or non-steroidal anti-inflammatories for the treatment of osteoarthritis.

The BH3 only protein, BH3 only protein fragments, or BH3 only protein encoding nucleic acids of the present invention can be formulated according to known methods to prepare pharmaceutically useful compositions. By way of example, PUMA is discussed herein. The pharmaceutical compositions can be formed by combining PUMA in admixture with a pharmaceutically acceptable carrier vehicle. Therapeutic formulations are prepared for storage by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions.

Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or PEG. Slow release polymer formulations are particularly preferred. Transdermal formulations are also preferred.

The route of administration is in accord with known methods, e.g., injection, intraperitoneal, intracerebral, intramuscular, intraocular, intraarterial or intralesional routes, topical administration, or by sustained release systems. Additionally, BH3 or nucleic acids encoding BH3 can be administered to cells and then transplanted into the individual. The cells can be autogenic, allogenic, syngenitic or xenogenic, and are preferably autogenic.

Dosages and desired drug concentrations of pharmaceutical compositions of the present invention may vary depending on the particular use envisioned. The determination of the appropriate dosage or route of administration is well within the skill of an ordinary physician. Animal experiments provide reliable guidance for the determination of effective doses for human therapy. In vivo examples are provided herein. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

Administration of the BH3 only protein in accordance with the present invention is preferably to a site of inflammation or potential inflammation. Administration can be in conjunction with a transdermal formulation, injected or applied topically directly to a site, i.e., an open wound or surgical site. In a preferred embodiment, BH3 only protein is administered. In another embodiment, nucleic acid encoding BH3 only protein is administered, preferably using a viral vector.

Use of Pharmaceutical Compositions

Once a nucleic acid vector or other agent for activating BH3 protein has been optimized, preclinical development typically involves testing in a suitable animal model.

Animal models for arthritis are reviewed by A. Bendele in J Musculoskelet Neuronal Interact. 2001 Jun.;1(4):377-85. Experimental arthritis can be induced in the rat by the injection an adjuvant such as Freund's (CM Pearson, Proc Soc Exp Biol Med. 1956 Jan.;91(1):95-101; Tas, Firestein et al., Hum Gene Ther.2006 Aug.;17(8):821-32). Another standard model for rheumatoid arthritis is collagen-induced arthritis (Courtenay et al., Nature 283:666, 1980; Gerlag et al., J Immunol 165:1652, 2000). There is also a TNFA transgenic mouse model of inflammatory arthritis (Li et al., Springer Semin Immunopathol. 2003 Aug.;25(1):19-33). Osteoarthritis can be induced in New Zealand white rabbits by the excision of the medial collateral ligament plus medial meniscectomy. Before initiating studies in a particular model, the user is advised to ensure that there isn't already a high endogenous expression of PUMA or other proapoptotic BH3 proteins.

Having identified a suitable model, the experimental condition is treated with a scaled dose of the test therapeutic composition (e.g., 50 μL into the joint of a rat). The clinical signs of the condition can be determined on an ongoing basis using a scaled scoring system appropriate for the model. Histology samples can be examined for standard pathology, and also for increased expression or activity of the intended BH3 protein by immunocytochemistry, or (at the mRNA level) by real-time PCR. Dose-response and timing studies will guide the user towards an optimal administration protocol. Also worthy of investigation is duration of BH3 protein expression in the target tissue, and expression in non-articular sites.

Human clinical trials are done according to the requirements of national regulatory agencies. In the evaluation of the compositions of this invention for treating rheumatoid arthritis, admission criteria might be patients with one major active (inflamed) joint who have not responded to (or have declined) steroid injection. In a dose escalation study, once reaching the highest tolerable dose, patients could undergo synovial biopsy for evaluation of pathology and BH3 protein expression. In addition to tissue endpoints, assessment could include MRI or ultrasound imaging. Alternatively, patients scheduled for total joint replacement could be treated before the procedure, and the entire joint could be taken to evaluate effects on cartilage and synovium. Subsequent efficacy studies could be done in a controlled trial (e.g., 1 placebo and 2 dose groups, with 30-40 per group).

The clinical condition of primary interest for treatment according to this invention is rheumatoid arthritis. This is because of the extensive impairment of natural apoptosis in arthritic joints (Yamanishi et al., Arthritis Res Ther. 2005;7(1):R12-8). Osteoarthritis is another potential indication, as are other conditions which can have inflamed joints as part of their pathology—perhaps psoriatic arthritis, juvenile arthritis, Reiter's Syndrome, arthritis associated with ulcerative colitis, Whipple's disease, arthritis associated with granulomatous ileocolitis, Behcet's disease, systemic lupus erythematosis, Sjogren's syndrome, and mixed connective tissue disease. Another potential indication is periodontal disease, in which case the composition would be formulated for administration to the gums or periodontal surface using an appropriate dental device.

Particularly suitable for treatment are human patients having a joint that is inflamed or affected out of proportion from most of the others. Administration is typically by direct injection at or near the synovial space in the affected joint or tissue.

In standard clinical practice, patients are monitored for improvement in the symptoms and signs of their disease. Effective modulation of synovitis is critical when attempting to improve the condition, prevent joint damage, and maintain function. Accordingly, the systematic evaluation of changes in synovial tissue after commencing treatment enables identification of an early therapeutic effect, using relatively small numbers of patients (Bresnihan, Firestein et al., J Rheumatol. 2005 Dec.;32(12):2481-4).

Quality control of pharmaceutical compounds before commercial distribution will involve verifying the sterility and activity of the compound, for example, by contacting an FLS with the composition, and determining whether apoptosis is induced in the cells as a consequence thereof. A medicament of this invention is typically packaged for commercial distribution in a suitable container accompanied by or associated with written information about its intended use, such as the condition to be treated, and aspects of dosing and administration.

Implementation of many aspects of the invention will involve conventional techniques of molecular biology, microbiology, recombinant DNA, and immunology.

Reference books for molecular genetics and genetic engineering include the current editions of Molecular Cloning: A Laboratory Manual, (Sambrook et al., Cold Spring Harbor); Gene Transfer Vectorsfor Mammalian Cells (Miller & Calos eds.); and Current Protocols in Molecular Biology (F. M. Ausubel et al. eds., Wiley & Sons). Cell biology, protein chemistry, and antibody techniques can be found in Current Protocols in Protein Science (J. E. Colligan et al. eds., Wiley & Sons); Current Protocols in Cell Biology (J. S. Bonifacino et al., Wiley & Sons) and Current Protocols in Immunology (J. E. Colligan et al. eds., Wiley & Sons.). References for the components of the apoptotic pathways and their use include Death Receptors in Cancer Therapy (W. S. El-Deiry, ed., Humana Press, 2005); Apoptotic Pathways as Targets for Novel Therapies in Cancer and Other Diseases (M. Los & S. B. Gibson eds., Springer, 2005); and Puma (T. L. Gragg, Surge Publishing, 2006). Formulation of gene therapy vectors for pharmaceutical use is described in Gene Therapy and Gene Delivery Systems (Schaffer & Zhou eds., Springer, 2005). Principles for the use of medicaments in the treatment of inflammatory conditions are described in Current Rheumatology: Diagnosis & Treatment (John B. Imboden et al., McGraw-Hill Medical, 2004); and Rheumatology (M. Hochberg et al. eds., C. V. Mosby, 2003).

The examples that follow are provided by way offurther illustration, and are not meant to limit the claimed invention.

### EXAMPLES

### Example 1:

### Materials and methods

Synovial tissue samples were obtained from patients with osteoarthritis (OA) and rheumatoid arthritis (RA) at the time of joint replacement. The diagnosis of RA conformed to the 1987 revised American College of Rheumatology criteria (Arnett et al., Arthritis Rheum (1988) 31:315-324). Normal human spleen tissue was provided by the UCSD Cancer Center, San Diego tissue bank. The studies were approved by the University of California, San Diego, Human Subjects Research Protection Program. FLS used for experiments were prepared from enzymatically dispersed synovial tissue by treating the tissues with 1 mg/ml collagenase and culturing in DMEM supplemented with 10% fetal calf serum (FCS), penicillin, streptomycin, and L-glutamate. Cell lines were used from the 3rd through 9th passage, when they are a homogenous population of fibroblast like cells. Normal Human skin fibroblasts were purchased from Cell Applications (San Diego, Calif.). p53+/+and p53−/−murine synoviocytes were obtained as previously described from DBA/1J wild type mice (Bar Harbor, ME) and DBA/IJ p53−/−mice.

Affinity-purified rabbit polyclonal anti-p53 (for immunohistochemistry), mouse monoclonal anti-p53 (for Western blot), and rabbit polyclonal antibodies for p21 and hemagglutinin (HA) were purchased from Santa Cruz Biotechnology (Santa Cruz, Calif.). Anti-mouse and anti-rabbit IgG secondary antibodies were purchased from Cell Signaling Technology, Inc. (Beverly, Mass.). Rabbit anti-PUMA polyclonal antibody was purchased from ProSci, Inc (Poway, Calif.).

Scrambled RNA and p53 siRNA were purchase from Dharmacon Research, Inc. (Lafayette, Colo.). Recombinant adenovirus expressing human wild-type-53 gene (Ad-p53) and recombinant adenovirus expressing β-galactosidase gene (Ad-βGal) (Introgen Therapeutics, Austin, Tex.) were used to infect cells at an approximate multiplicity of infection of 100 for 24 hr. Plasmids encoding hemagglutinin (HA)-tagged, full-length PUMA expression vector (HA-PUMA) and HA-tagged, PUMA expression vector with a deletion of a the BH3 domain (HA-PUMAdBH3) were kindly provided by Dr. Bert Vogelstein (Johns Hopkins Oncology Center, Baltimore, Md.). Empty vector (pCEP4) (Invitrogen Life Technologies, Carlsbad, Calif.) was used as a negative control. FLS transfection was performed by using the Amaxa HUMAN DERMAL FIBROBLAST NUCLEOFECTION KIT™ (NHDF-adult) (Amaxa Biosystems, Gaithersburg, Md.) as described previously (Inoue et al. (2005), supra). R213* encoding mutant p53 was isolated from a patient with RA and has been previously characterized as dominant negative (14). Bax-lux (BF72-2 PGL3) is a reporter construct containing the p53 responsive promoter for bax with the luciferase cDNA (14). The control construct contains the βgal cDNA and the cytomegalovirus (CMV) promoter in pCL. Cells were transfected as above, with program U-23 for human FLS. Murine FLS were transfected using MOUSE EMBRYONIC FIBROBLASTS KIT™ (MEF1) with program T-20. 2-10×10⁵ cells were transfected with siRNAs, cDNAs, or control plasmids in each reaction.

Cultured FLS were washed with PBS and protein was extracted using lysis buffer (50 mM HEPES, 150 mM NaCl, 1% Triton-X 100, 10% glycerol, 1 mM MgCl₂, 1.5 mM EDTA (pH 8.0), 20 mM β-glycerophosphate, 50=mM NaF, 1 mM Na₃VO₄, 10 μg/ml aprotinin, 1 μM pepstatin A, and 1 mM PMSF). The protein concentrations were determined using the DC protein assay kit (BioRad, Hercules, CA). Whole cell lysates containing 50 μg of protein were fractionated on 12% SDS-PAGE and transferred to a nitrocellulose membrane. The membrane was blocked with TBS and 0.1% Tween™ 20 (TBST) containing 5% nonfat milk for 1 hr at room temperature followed by incubation with the appropriate antibody at 4° C. overnight. The membrane was washed three times and incubated with HRP-conjugated secondary antibody for 1 hr. Immunoreactive protein was visualized with chemiluminescence using Kodak X-AR film (Eastman Kodak, Rochester, N.Y.). Alternatively, Western blot analysis was performed on ST and FLS with antibodies against PUMA (ProSci), p53, p21, actin, and HA-probe (Santa Cruz Biotechnology, Santa Cruz, Calif.), according to manufacturer's instructions as previously described (Inoue et al. (2005), supra). Quantitative real-time PCR was performed to determine relative PUMA mRNA levels in tissues and cells using the Gene Amp 5700 Sequence Detection System (Applied Biosystems, Foster City, Calif.)(Boyle et al., Arthritis Res Ther (2003) 5:R352-360).

siRNA-transfected cells for immunostaining were cultured in 4-well chamber slides at 4.0×10⁴ cells/well. The were then fixed with methanol, permeablized with 0.05% Triton-X 100 and blocked with 10% human serum. The fixed cells were incubated with anti-p53 antibody or matched control antibody overnight at 4° C. Endogenous peroxidase was then depleted with 0.1% H₂0₂ and 0.1% NaN₃. The cells were then washed and stained with biotinylated secondary antibody anti-mouse or anti-rabbit IgG and Vectastain ABC and developed using diaminobenzinide (Vector, Burlingame, Calif.). Other immunohistochemistry was performed as previously described using anti-PUMA antibody (ProSci, Poway, Calif.) (Elices et al., J Clin Invest (1994) 93:405-416).

Transduced FLS were suspended in 0.4% Trypan Blue in PBS, and counted using a hemocytometer. The cells that excluded the dye were considered viable. Apoptosis was determined using the CELL DEATH DETECTION ELISAPLUS KIT™ (Roche, Mannheim, Germany) to detect histones according to the manufacturer's instruction. Each measurement was performed in duplicate, and the results presented as the fold induction compared with control. Activation of caspase 3 was determined by immunohistochemistry using specific antibody against active caspase-3 (BD PharMingen, San Jose, Calif.).

Alamar Blue assays incorporate flurometriuc/colormetric growth indicator based on detection of metabolic activity. 3×10³ FLS were plated into 96-well plate after siRNA transfection. At various time points, medium was replaced by DMEM without phenol red supplemented with 10% Alamar Blue. After incubating for 4 hours at 37° C., fluorescence was measured with a microplate reader at 530 nm excitation wavelength and 590 emission wavelength. The number of cells is expressed as relative fluorescence units (RFU).

Cells were transfected with the Bax-luciferase reporter construct, the CMV-βgal reporter construct, and control empty plasmid to equalize the amount of plasmid in each reaction. To quantify luciferase expression, cells were washed twice with PBS and incubated with 100 μl of lysis buffer. Cellular debris was removed by centrifugation and 20 μL of supernatant were assayed by adding 100 μL of luciferase assay reagent (Luciferase Reporter Gene Assay, Roche Applied Science, Penzberg, Germany). Luciferase activity was measured in relative light units using a luminometer. The results were normalized to βgal expression, the clarified cell lysates were incubated with CPRG substrate (High Sensitivity βgalactosidase Assay, Stratagene Corp., La Jolla, Calif.). The reaction was incubated at 37° C. for 30 min and Pgal activity was determined using a microtiter plate reader at a wavelength of 570 nm.

### Example 2:

### PUMA Regulation and Proapoptotic Effects in Fibroblast-like Synoviocytes

In this example, experiments were performed to assess PUMA expression in synovial tissue obtained from patients with rheumatoid arthritis.

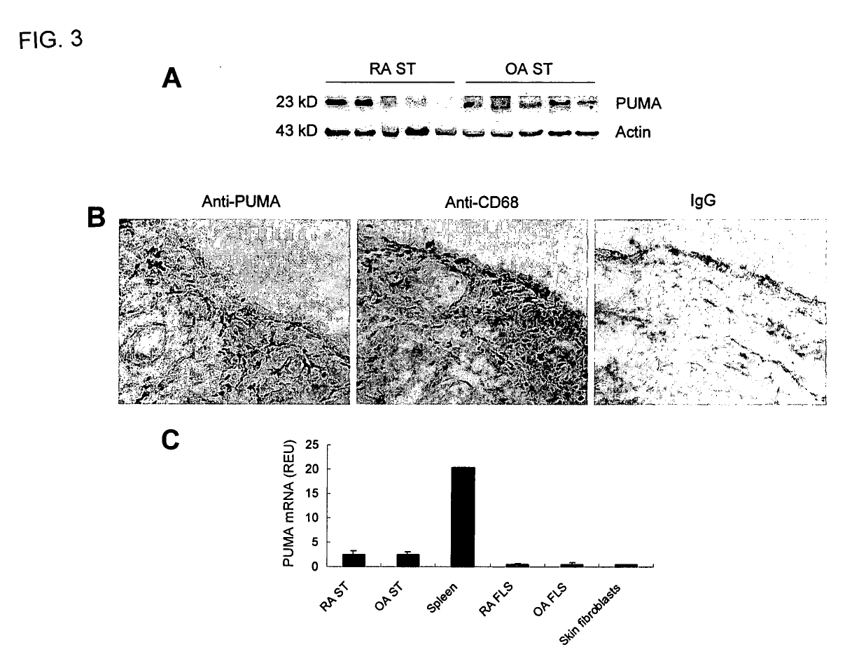

FIG. 3 shows PUMA protein and gene expression in RA synovium and FLS. FIG. 3(A): Total protein from RA (n=5) and OA (n=5) ST was extracted and Western blot analysis was performed to detect PUMA and β-actin expression. FIG. 3(B): A representative example of immunohistochemistry to detect PUMA is shown for RA ST. Although expression was low, when PUMA was detected in RA synovium it was mainly expressed the synovial sublining area. For comparison to demonstrate the intimal lining in the same tissue, CD68 expression in the lining cells is also shown. A serial section with an isotype matched control antibody (IgG) was negative. FIG. 3(C): Total RNA was isolated from RA ST (n=16), OA ST (n=15), normal human spleen tissue, RA FLS (n=5), OA FLS (n=5), and normal skin fibroblasts (n=4) and quantitative real-time PCR was performed to measure PUMA MRNA expression. Data are mRNA level normalized to GAPDH expression and shown as relative expression units. Low levels of PUMA MRNA were detected in RA and OA ST (no difference was observed between RA and OA ST), but lower than ST; P<0.05 compared with an immunologically active tissue (normal spleen tissue), which contained 8 fold more PUMA MRNA than synovium. PUMA mRNA expression in FLS and skin fibroblasts (RA FLS, OA FLS, and skin fibroblasts were similar) were significantly lower than ST (P<0.05), which is consistent with the immunohistochemistry data showing minimal expression in the intimal lining.

Because only low levels of PUMA were detected in the rheumatoid synovial intimal liming and FLS, the effect of p53 was on PUMA expression was determined.

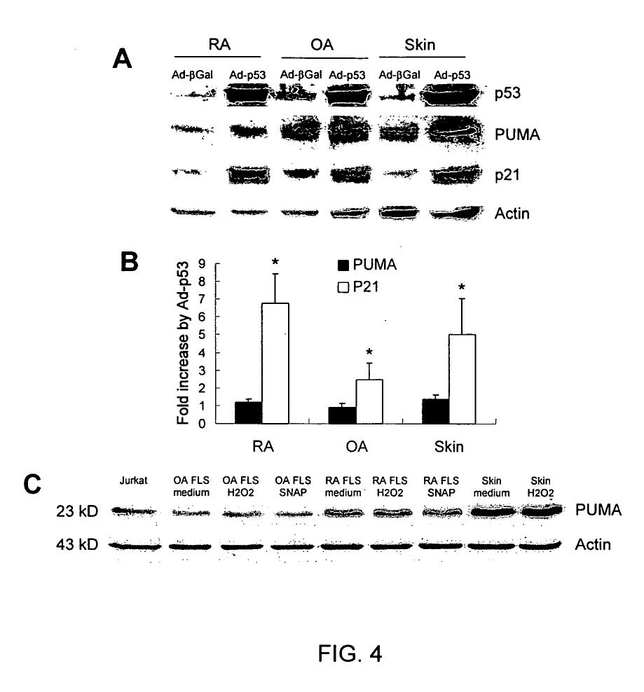

FIG. 4 shows Induction of p21 but not PUMA by Ad-p53. Western blot analysis was performed in RA (n=3), OA (n=3) FLS and normal skin fibroblasts (n=3) to detect PUMA or p21 protein expression after Ad-p53 or Ad-βGal infection for 24 hr. FIG. 4(A): A representative Western blot from 3 independent experiments is shown. FIG. 4(B): Data are presented as fold change of β-actin normalized PUMA or p21 expression level in Ad-p53 infected cells compared with Ad-pGal-infected cells as controls. Ad-p53 significantly increased p21 protein expression (*P<0.05), but PUMA was not induced by Ad-p53. FIG. 4(C): RA FLS, OA FLS, and skin fibroblasts were exposed to 0.06 mM hydrogen peroxide or 1 mM SNAP for 12 hrs and analyzed for PUMA expression by Western blot. Note that PUMA expression was not increased by exposure to reactive oxygen or the nitric oxide donor. Jurkat cells are included as a positive control.

Thus, in spite of induction of p53 expression, PUMA expression remained low in RA and OA FLS as well as in skin fibroblasts(1.18±0.21, 0.91±0.22, and 1.38±0.21 fold increase by Ad-p53 compared with Ad-pGal, respectively). On the other hand, FLS and skin fibroblasts infected with Ad-p53 showed significant increase of the p53-regulated p21 expression (6.8+1.7, 2.4V1.0, and 5.0±2.1 fold increase, respectively; P<0.05 for each).

p21, plays an essential role in growth arrest after DNA damage (Gartel and Tyner, Mol Cancer Ther (2002) 1:639-649), and is a negative regulator of p53-dependent apoptosis. For example, repression or elimination of p21 expression by antisense, E1A, or triptolide enhances the apoptotic effect of p53, where as overexpression of p21 suppresses p53-dependent apoptosis (Gartel and Tyner Mo Cancer Ther (2002) 1:639-649). As shown above, p53 overexpression induced p21 but not PUMA or apoptosis. While it is clear that the present system demonstrates that p53 alone was not sufficient to activate programmed cell death, based on collagen-induced arthritis, overexpression of p53 might be beneficial in RA through induction of p21, even if PUMA is not induced.

Because p53 overexpression in FLS and skin fibroblasts did not induce PUMA, the apoptotic effect of Ad-p53 was determined for these cells. FLS and skin fibroblasts were infected with Ad-p53 or Ad-βGal (n=3 each) and apoptosis was measured using ELISA that detects cytoplasmic histone-associated DNA fragments. Ad-p53 did not significantly increase apoptosis in RA and OA FLS or skin fibroblasts (1.25±0.18, 1.08±0.43, and 1.14±0.39 fold induction of DNA fragmentation, respectively; P>0.10 for each).

Because p53 overexpression did not induce PUMA or apoptosis in FLS, whether overexpression of PUMA could bypass p53 and induce synoviocyte programmed cell death was evaluated. RA FLS were transfected with pCEP4, HA-PUMA, or HA-PUMAdBH3. Transfection was confirmed by demonstrating PUMA and PUMAdBH3 protein by Western blot analysis after 12 hrs.

FIG. 5 shows that apoptosis was indeed induced in RA FLS by PUMA overexpression. FIG. 5(A), RA FLS (n=3) were transfected with pCEP4, HA-PUMA, or HA-PUMAdBH3. After 24 hr, Trypan Blue exclusion was determined. Data are presented as the percentage of non-viable cells. HA-PUMA-transfected FLS showed significantly more dead cells compared with pCEP4 or HA-PUMAdBH3-transfected cells (*P<0.01). FIG. 5(B): Comparison of apoptosis induced by pCEP4, HA-PUMA, or HA-PUMAdBH3 in RA (closed bar) and OA (open bar) FLS lines 24 hr after transfection. The extent of PUMA-induced apoptosis was the same in both cell lines. Similar results were also observed after 48 hr; FIG. 5(C): RA FLS (n=3) were transfected with pCEP4, HA-PUMA, or HA-PUMAdBH3, and after 12 hr, DNA fragmentation was measured by ELISA. The fold induction of DNA fragmentation in HA-PUMA or HA-PUMAdBH3-transfected FLS is shown relative to the control value (pCEP4-transfected FLS). Significant induction of DNA fragmentation was noted in HA-PUMA-transfected cells compared with pCEP4-transfected cells (*P<0.05). FIG. 5(D): pCEP4, HA-PUMA, or HA-PUMAdBH3-transfected FLS were cultured in chamber slides for 12 hr. The cells were then immunostained for activated caspase-3. Data presented as percentage of activated caspase-3 positive cells. PUMA-transfected FLS showed significantly more of activated caspase-3 positive cells compared with pCEP4 or HA-PUMAdBH3-transfected cells (*P<0.01).

Discussion

Insufficient apoptosis could potentially contribute to synovial intimal lining hyperplasia in RA, thereby enhancing local production of pro-inflammatory mediators and metalloproteinases. Several studies suggest that anti-apoptotic genes are underexpressed in the rheumatoid synovium and might contribute to this phenomenon. However, the relative lack of synoviocyte death despite expression of the p53 tumor suppressor gene is also poorly understood. One possibility is that the p53 gene is abnormal in RA, although only a limited percentage of the p53 cDNA pool contains mutations (Firestein et al., Proc Natl Acad Sci U S A 1997;94: 10895-900). Alternatively, activation of the downstream machinery for apoptosis might not function effectively in synoviocytes. Surprisingly, the present study suggests that PUMA, a major effector of p53-mediated cell death, is not induced by p53 in cultured FLS, which could explain some of these observations.

These studies demonstrated that low levels of PUMA are expressed in RA and OA synovial tissue. These results contrast with normal spleen cells, which express markedly higher amounts. Of interest, very little immunoreactive PUMA was detected in the synovial intimal lining, which is a major site of p53 expression. Very low PUMA expression in cultured FLS was also observed, which is consistent with immunostaining studies suggesting that the gene is mainly expressed in sublining cells. These data suggest that synoviocytes exhibit low PUMA gene expression in response to apoptotic stimuli or p53 in the synovial intimal lining.

To address this possibility, we investigated PUMA expression in response to p53 in FLS. Surprisingly, p53 overexpression did not induce PUMA in FLS. This was not due to global dysfunction of the p53 machinery because p21 levels were significantly increased in the same cells. Consistent with the lack of PUMA expression, Ad-p53 failed to induce apoptosis in FLS. The results suggest that the response to p53 in FLS might be activation of the cell-cycle arrest pathway via p21 induction rather than PUMA-mediated apoptosis. This cellular response to p53 is not unique to RA FLS, because similar response was observed in OA FLS and normal skin fibroblasts.

p21, a cyclin-dependent kinase inhibitor, plays an essential role in growth arrest after DNA damage (Gartel et al., Mol Cancer Ther 2002; 1:639-49). It is also a negative regulator of p53-dependent apoptosis. Repression or elimination of p21 expression by antisense, EIA, or triptolide enhances the apoptotic effect of p53, whereas overexpression of p21 suppresses p53-dependent apoptosis (Gartel et al.). The reason that p53 overexpression induces p21 but not PUMA or apoptosis in FLS is unknown. Tissue and cell type-specific selectivity of p53 in transactivation of downstream genes has been reported, including cells in which p53 induces p21 but not PUMA after doxorubicin treatment or irradiation (Fei et al., Cancer Res 2002;62:7316-27). The failure of efficient apoptosis by Ad-p53 in FLS demonstrated in this study differs in some respects with a previous report in which Ad-p53 reduced FLS viability (Yao et al., Mol Ther 2001;3:901-10). The reason for such a discrepancy is not clear but might relate to culture conditions or other uncertain variables. Nevertheless, it is clear that in this system p53 alone was not sufficient to activate programmed cell death. However, based on collagen-induced arthritis studies, overexpression of p53 might be beneficial in RA though the induction of p21 even if PUMA is not induced (Perlman et al., J Immunol 2003;170:838-45).

The relative absence of PUMA, which is a downstream effector of p53-mediated apoptosis, could contribute to low apoptosis in the intimal lining. PUMA interacts with anti-apoptotic Bcl-2 protein family members such as Bcl-xL and induces mitochondrial dysfunction though Bax (Jeffers et al., Cancer Cell. 2003;4:321-8). The BH3 domains are essential for its pro-apoptotic activity (Yu et al., Mol Cell 2001;7:673-82), which was also confirmed in this study because PUMAdBH3 did not induce cell death p53 expression is not necessarily required for PUMA to regulate apoptosis, as recently demonstrated in a study showing that adenoviral gene transfer of PUMA causes in massive apoptosis of malignant glioma cells regardless of p53 status (Ito et al., Hum Gene Ther 2005;16:685-98). PUMA was superior to p53, caspase-6 and caspase-8 with regard to the ability to induce cell death.

This example demonstrates that PUMA expression is low in the synovial intimal lining and cultured FLS. Gene transfer with PUMA rapidly increased apoptosis in FLS even though p53 overexpression was ineffective. This supports PUMA as a potential therapeutic agent for inducing synoviocyte death in rheumatoid arthritis.

### Example 3

### PUMA Regulation and Proapoptotic Effects in fibroblast-like Synoviocytes

Experiments were performed to determine whether siRNA could knock down p53 expression in cultured FLS.

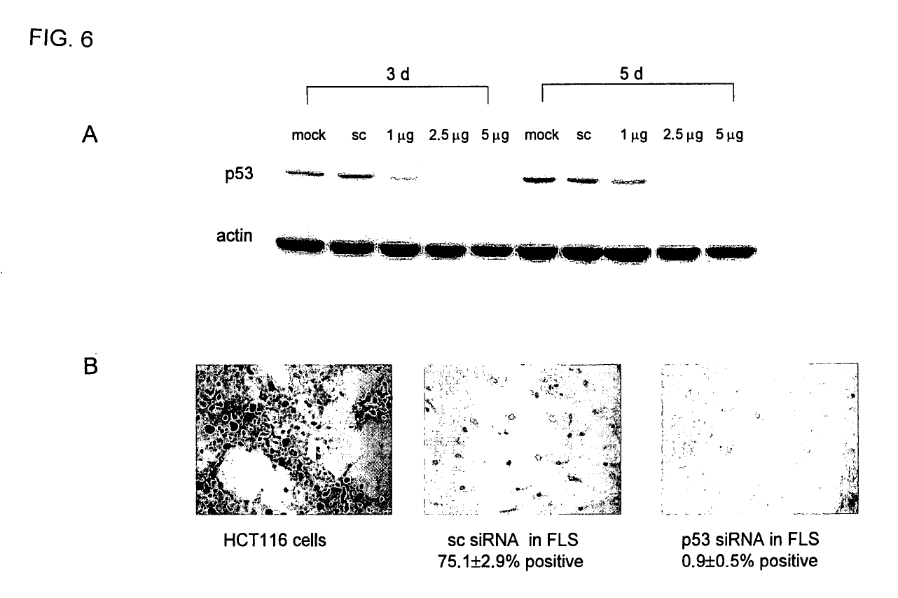

FIG. 6 shows a representative time course of the effect of siRNA p53 knockdown on cultured FLS. FIG. 6(A): Western blot analysis. Cultured FLS were transfected with 1, 2.5, or 5 μg of siRNA or non-silence scrambled siRNA (sc). Mock transfected cells were treated in the same manner except that no siRNA was added. FLS were then incubated for 3 or 5 days and Western blot analysis was performed. FIG. 6(B): Irnmunohistochemistry staining of p53 protein expression in RA FLS. Transfected FLS were seeded into 4-well chamber slides, cultured for 5 days, and evaluated by immunohistochemistry. Manse percentage of p53-positive cells is shown.

The residual expression of p53 protein was approximately 8-10%, 5%, and 1-2% for 1, 2.5, and 5 μg of siRNA as determined by Western blot analysis. Immunohistochemistry also demonstrated a marked decrease in the percentage of p53 positive cells after siRNA transduction. The percentage of cells with detectable p53 protein was 7.5±2.9% for scrambled siRNA, 2.8±0.65 for 1 μg of p53 siRNA, and 0.9±0.5% for 5 μg of siRNA.

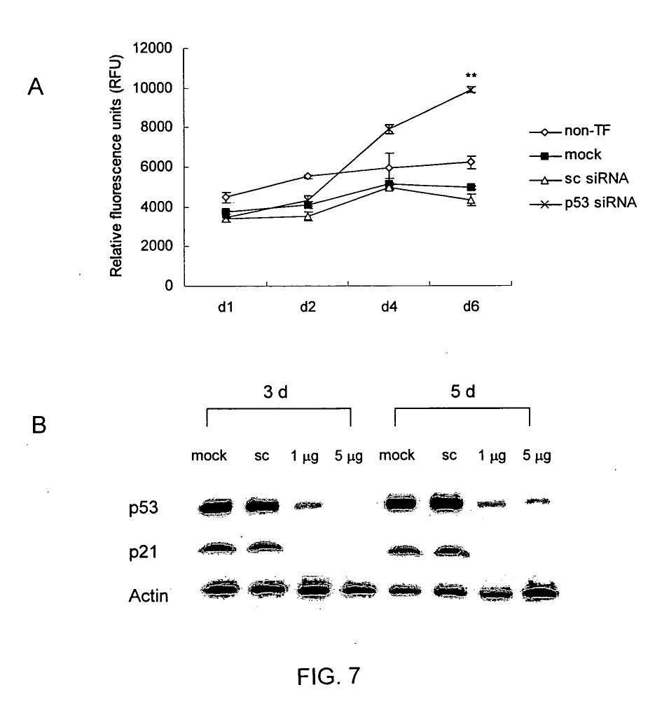

FIG. 7 shows the effect of p53 siRNA on FLS function. FIG. 7(A): cell growth. p53 was knocked down using siRNA and cell growth was determined using an Alamar Blue assay in triplicate wells (n=3 separate cell lines). The relative number of cells is detected by arbitrary fluorescence units, and the fold induction of RFU in p53 siRNA transfected FLS is shown relative to the fluorescence value of mock cells. Similar results were observed with 5 μg of siRNA. P<0.001 for p53 siRNA transfected cells compared with scrambled siRNA. RFU—relative fluorescence units. FIG. 7(B): p21 protein expression. P53 siRNA was knocked down using siRNA and p21 expression was determined by Western blot analysis. P21 expression decreased in cells with deficient p53 expression (n=2 separate cell lines). These data indicate that p53 knockdown leads to functional alternations consistent with p53 deficiency.

To determine if p53 is required for PUMA-induced apoptosis, FLS were transduced with p53 siRNA. After p53 expression reached its nadir three days later, cells were transfected with PUMA cDNA, PUMAbBH3, or empty plasmid pCEP4.

FIG. 8 illustrates the effect of PUMA on p53-deficient human FLS. Cultured FLS were transfected with siRNA and then 3 days later, with 100 μg of PUMA, PUMAdBH3, or pCEP4. FIG. 8(A):. DNA fragmentation as determined by histone release. Histone release was measured by ELISA in samples collected 9 hr after the second transfection. The fold induction of DNA fragmentation in PUMA plasmids-transfected FLS is shown relative to the control value of pCEP4-transfected cells. *P<0.05, n=3. FIG. 8(B):. Cell viability. Trypan Blue exclusion was evaluated at 24 hr after cDNA transfection in 2.5 μg of siRNA-treated FLS. Mock (stippled square), PUMA (black square), PUMAdBH3 (gray square), and pCEP4 (open square). Data are presented as the percentage of non-viable cells. FIG. 8(C): Cell viability. PUMA (black square) and pCEP4 (open square), only. *P<0.0, **P<0.001, n=3.

Thus, histone release or cell viability were similar in scrambled siRNA- and p53 siRNA-transfected cells. PUMAdBH3, which lacks the BH3 domain and does not induce apoptosis, had no effect on cell viability. Because Bax is thought to play a major role in PUMA-mediated apoptosis, Bax transcription was also examined in FLS with p53 deficiency. Bax promoter activity was similar in cells transducer with scrambled siRNA or p53 siRNA.

Small amounts of residual p53 might contribute to PUMA apoptosis in the siRNA studies, so the experiments were repeated in p53+/+and p53−/−murine synoviocytes.

FIG. 9 illustrates the effect of PUMA on p53+/+and p53−/−murine FLS. Passage 6 murine p53+/+and p53−/−FLS were transfected. FIG. 9(A): Cell viability by Trypan Blue staining (*P<0.05, n=3). FIG. 9(B): Histone protein release (*P<0.05, n=3) to evaluate PUMA-induced apoptosis in both wild type and p53 knockout FLS. Thus, overall cell viability was decreased in the transfected cells, even with the control plasmid. However, the effect of PUMA compared with PUMAdBH3 or empty vector was the same in p53+/+and p53−/−synoviocytes.

Because some RA synoviocytes might express dominant negative p53 protein, whether PUMA could induce cell death in the presence of mutant p53 was determined. A known dominant negative gene that was isolated form an RNA patient (R213*) was used for these experiments. Sequential transfection of cultured human FLS with R213* and followed PUMA two days later was performed.

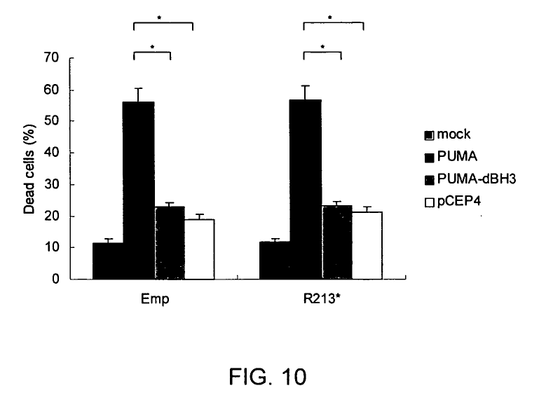

FIG. 10 illustrates the effect of PUMA on human FLS containing mutant p53. Human FLS were transfected with dominant negative p53 cDNA R213* or controls followed by PUMA cDNA. *P<0.05, n=3. Thus, PUMA effectively induced apoptosis in FLS even in the presence of a dominant negative p53.

These data confirm that PUMA-induced apoptosis in synoviocytes does not require p53 and that PUMA gene transfer could be effective regardless of the p53 status of the synovium. Thus, intra-articular gene transfer should decrease and depopulate the synovial intimal lining, where local therapy could debulk the synovium in RNA and serve as an alternative to synovectomy or intra-articular corticosteroids.

Although the invention has been described with reference to the above examples, it will be understood that modifications and variations are encompassed within the invention

The invention claimed is:

1. A pharmaceutical composition comprising a BH3-only proapoptotic Bcl-2 protein, or a nucleic acid vector for expressing said protein, formulated in an excipient for administration into an inflamed joint in a mammalian subject.

2. The composition of claim 1, comprising a nucleic acid vector having an encoding sequence for said BH3-only proapoptotic Bcl-2 protein operably linked to a promoter that causes expression of said protein in fibroblast like synoviocytes (FLS).

3. The composition of claim 1, wherein said vector is an adenovirus vector.

4. The composition of claim 1, wherein said BH3-only proapoptotic Bcl-2 protein is PUMA (the p53 upregulated modulator of apoptosis).

5. The composition of claim 1, wherein said BH3-only proapoptotic Bcl-2 protein is at least 90% identical to SEQ. ID NO:2 or SEQ. ID NO:3, or a fragment of either of said sequences that promotes apoptosis in mammalian cells.

6. The composition of claim 1, wherein said BH3-only proapoptotic Bcl-2 protein is encoded by a nucleic acid that hybridizes under stringent conditions to SEQ. ID NO: 1, and promotes apoptosis in mammalian cells.

7. The composition of claim 1, packaged with information for the administration of the composition into an inflamed joint.

8. The composition of claim 1, packaged with information for use of the composition for treating rheumatoid arthritis or osteoarthritis.

9. A method for preparing the pharmaceutical composition of claim 1, comprising formulating a BH3-only proapoptotic Bcl-2 protein, or a nucleic acid vector encoding said protein, for administration into an inflamed joint in a human subject.

10. A method for killing a fibroblast-like synoviocyte (FLS) in vitro, comprising contacting said FLS with a BH3-only proapoptotic Bcl-2 protein, or a nucleic acid vector encoding said protein.

11. A method for determining whether an agent can induce expression of a BH3-only proapoptotic Bcl-2 protein in a fibroblast-like synoviocyte (FLS), comprising contacting an FLS in vitro with said agent, and determining whether apoptosis is induced in said FLS as a consequence thereof.

12. A method for determining whether an agent that promotes expression of a BH3-only proapoptotic Bcl-2 protein is suitable for preparation of a medicament for treating an inflamed joint, comprising contacting an FLS in vitro with said agent, and determining whether said agent causes apoptosis of the FLS.

13. A quality control method for assessing a BH3-only proapoptotic Bcl-2 protein, or a nucleic acid vector encoding said protein, for use in treating an inflamed joint in a human subject, comprising contacting an FLS with said protein or nucleic acid vector, and determining whether apoptosis is induced in said FLS as a consequence thereof.

14. A method for causing apoptosis in synoviocytes and other inflammatory cells in vivo, comprising inducing expression of a BH3-only pro-apoptotic Bcl-2 protein in the cells.

15. A method for treating inflammation in a subject, comprising inducing expression of a BH3-only pro-apoptotic Bcl-2 protein in cells at or around a site of inflammation in the subject.

16. A method for treating arthritis in a subject, comprising administering to an inflamed joint in the subject an agent that promotes expression of a BH3-only pro-apoptotic Bcl-2 protein in synoviocytes.

17. The treatment method of claim 16, wherein expression of said protein is induced by administrating an adenovirus expression vector encoding said BH3-only proapoptotic Bcl-2 protein.

18. The treatment method of claim 16, wherein said BH3-only proapoptotic Bcl-2 protein is PUMA (the p53 uregulated modulator of apoptosis).
